(19) United States
(12) Patent Application Publication
(10) Pub. No.: US 2009/0023819 A1
(43) Pub. Date: Jan. 22, 2009

(54) Use of an Artificial Sweetener to Enhance Absorption of Nicotine

(76) Inventor: Anders Axelsson, Bjarred (SE)

Correspondence Address: FULBRIGHT & JAWORSKI, LLP, 1301 MCKINNEY, SUITE 5100, HOUSTON, TX, 77010-3095, US

(21) Appl. No.: 11/909,150

(22) PCT Filed: Mar. 17, 2006
(86) PCT No.: PCT/EP2006/002675

§ 371 Date: Sep. 19, 2008

(30) Foreign Application Priority Data
Mar. 22, 2005 (DK) PA 2005 00414

(51) Int. Cl. A61K 47/22

(52) U.S. Cl. 514/788

(57) ABSTRACT

The present invention relates to increased absorption of nicotine over the prior art. In particular the absorption of nicotine is enhanced after administration of a composition containing nicotine and a sweetener such as an artificial sweetener like saccharin to the oral mucosa in the form of a spray.

### FIELD OF THE INVENTION

The present invention relates to increased absorption of nicotine over the prior art. In particular the absorption of nicotine is enhanced after administration of a composition containing nicotine and a sweetener such as an artificial sweetener like saccharin to the oral mucosa in the form of a spray.

### BACKGROUND OF THE INVENTION

Smoking behavior is associated with serious health risks not only to the smoker but also to the people around him, who is exposed to passive smoking. To quit smoking has therefore been the expert's advice for many years. However, the smoker is addicted to nicotine, which makes quitting quite difficult for most smokers. Another way of nicotine administration than smoking has been employed in the efforts of helping smokers quitting their unhealthy habit. Several products employing e.g. oral or transdermal administration of nicotine are currently available for smokers wanting to quit smoking. Such administration is e.g. achieved by chewing gums, inhalators, patches or mouth sprays.

In spite of the availability of several nicotine compensation products such as those mentioned above, many smokers still do not find it easy to quit smoking and although the explanation hereto is probably a combination of multiple factors, two of them being the attained concentration of nicotine in the bloodstream and the rate at which nicotine is reaching the bloodstream providing the smoker with the desired effect.

Accordingly, there is a need to development methods or compositions that enable a faster on-set of the nicotine effect and a faster rise in the plasma concentration of nicotine after administration.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION OF THE INVENTION

In keeping with long-standing patent law convention, the words “a” and “an” when used in the present specification in concert with the word comprising, including the claims, denote “one or more.” As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The present invention relates to the increased absorption of nicotine over the prior art which is thought at the time of the application to be due to the co-administration of nicotine with a sweetener, such as an artificial sweetener, such as an artificial sweetener having a sulfonamide, such as e.g. saccharine or Ace-K, or the route of administration (i.e. directional spraying to administer nicotine and/or nicotine-sweetener (etc.) combination to the space between the gum and the cheek), or a combination of the route of administration and a sweetener, such as an artificial sweetener, such as an artificial sweetener having a sulfonamide, such as saccharine or Ace-K.

The present invention addresses the above-mentioned problems by providing compositions that include a sweetener that is believed to function as an absorption-enhancing agent. The present inventors have found that artificial sweeteners like e.g. saccharin seem to enhance the absorption of nicotine. However, the route of administration may also have impact on the absorption, cf. above.

As used herein, the term “absorption-enhancing agent” means an agent that enhances the absorption of nicotine, in particular the absorption rate, i.e. increases the rate by which nicotine reaches the bloodstream thereby resulting in a higher plasma concentration of nicotine in less time.

Accordingly, in one aspect the present invention relates to the use of an artificial sweetener comprising a sulfonamide group, or a salt, complex, derivative or solvate thereof, as an absorption enhancing agent for nicotine or a salt, complex, derivative or solvate thereof.

In the present context a salt, complex, derivative or solvate of a compound is therefore intended to include a pharmaceutically acceptable salt, complex, derivative or solvate of that compound in those cases where a composition according to the present invention is a pharmaceutical composition. However, it is contemplated that the teachings of the present invention can also be applied for administration via food and/or stimulants and in those cases the term “salt, complex, derivative or solvate of a compound” includes such salts, complexes, derivatives or solvates that are of food grade or normally acceptable to be used for stimulants.

In another aspect, the present invention provides a composition comprising a sweetener such as an artificial sweetener comprising a sulfonamide group, or a salt, complex, derivative or solvate thereof, and nicotine or a salt, complex, derivative or solvate thereof, wherein the sweetener such as the artificial sweetener and the nicotine is present in a weight ratio of from at least about 0.2 such as, e.g., at least about 0.3, at least about 0.4 or at least about 0.5, and the weight ratio is calculated as the ratio between the sweetener such as the artificial sweetener in the form of its sodium salt and nicotine in the form of the free base nicotine.

In a specific embodiment, a composition according to the present invention is a pharmaceutical composition.

The present invention provides improvements over prior art both with respect to attained maximum plasma-concentrations of nicotine and with respect to the rate of nicotine-absorption in the bloodstream.

To our knowledge the combination of saccharin and nicotine is only known from a mouth spray product called Quit, which is available since several years in South Africa. However, in this product saccharin is used as a sweetener and is present in very low concentrations (0.114% (w/v)) and the weight ratio, calculated as the ratio between saccharin in the form of its sodium salt and nicotine in the form of the free base nicotine, is 0.08.

The Quit mouth spray is available in different strengths so as to start the nicotine replacement therapy using the highest strength (2 mg nicotine per 2 sprayings) and then gradually decrease the dose of nicotine using the lower strength products until finally applying a product containing no nicotine. According to data once published on the Quit internet site the highest strength Quit mouth spray is bioequivalent to the lower strength of Nicorette nicotine gum (i.e. 2 mg).

However, as described herein the on-set of the effect obtained by use of a composition according to the present invention is faster than by use of Quit in an equivalent dose.

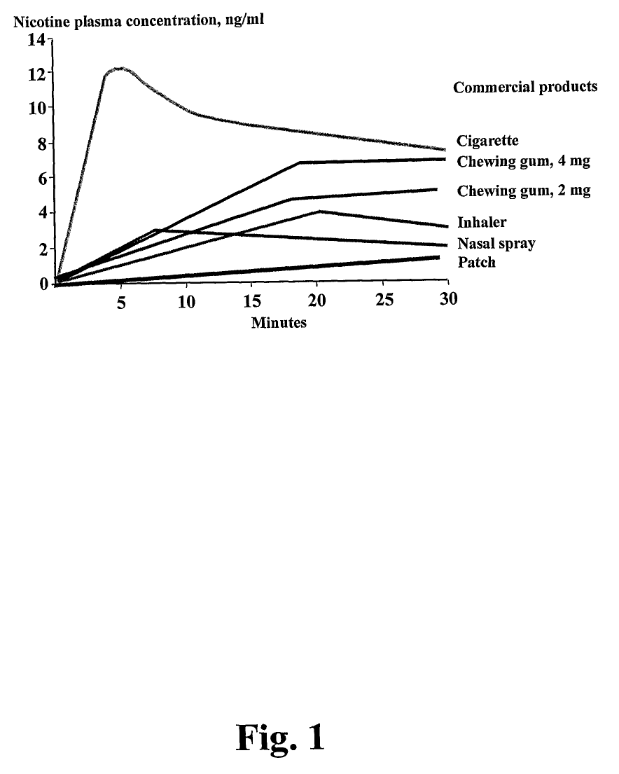

The inventors of the present invention have demonstrated a significant difference between the rate of nicotine-absorption in the bloodstream when smoking cigarettes as compared to several commercially available nicotine replacement products, the cigarettes providing the smoker with a faster rate of nicotine-absorption in the bloodstream than any of the nicotine replacement products (FIG. 1). The rate at which the nicotine reaches the bloodstream, is suspected to be an important factor for the success rate of quitting smoking: The smoker is used to a fast effect upon smoking and the slow effect of current nicotine replacement products, therefore does not provide the smoker with the same stimulation as a cigarette.

As mentioned above, in one aspect the present invention relates to the use of a sweetener such as an artificial sweetener, or a salt, complex, derivative or solvate thereof, as an absorption enhancing agent for nicotine or a salt, complex, derivative or solvate thereof. The absorption enhancing effect of the sweetener, for example an artificial sweetener, or salt, complex, derivative or solvate thereof, results in an increased absorption rate of nicotine, i.e. the rate at which nicotine reaches the bloodstream is increased resulting in higher plasma concentrations of nicotine in less time. Furthermore, the absorption enhancing effect of the artificial sweetener results in plasma levels of nicotine, which are comparable to the levels obtained upon smoking.

In an embodiment of the invention, the artificial sweetener for use according to the invention comprises a sulfonamide group. Suitable examples include saccharin or acesulfame (also known as acesulfame-K, acesulfame potassium) or salts, complexes, derivatives or solvates thereof.

As demonstrated in the Examples herein, a particular suitable artificial sweetener is saccharin. When used herein the term “saccharin” includes saccharin as well as salts, complexes, derivatives or solvates thereof. Of particular interest are alkaline or alkaline earth metal salts such as, e.g., saccharin sodium, saccharin potassium, saccharin calcium etc.

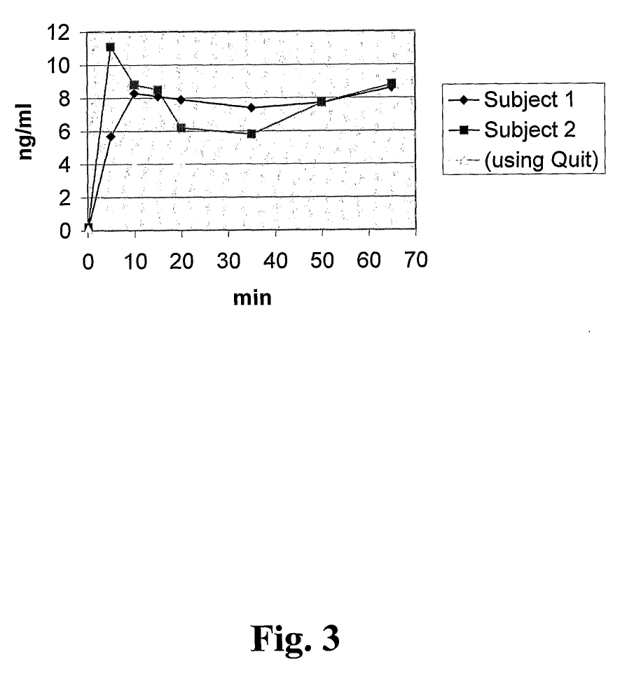

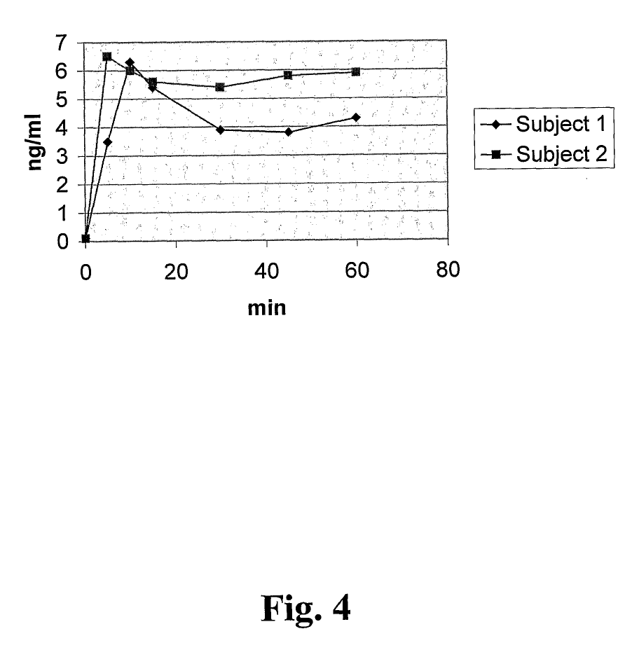

In the Examples herein the absorption-enhancing effect is demonstrated with saccharin and in the form of a solution for administration to the oral mucosa (see FIGS. 3 and 4). Data presented by the present inventors surprisingly indicate a correlation between saccharin concentration and the rate of nicotine-absorption to the bloodstream. Such effect of saccharin was not to be expected, since saccharin has an acidic reaction according to Handbook of Pharmaceutical Excipients pp 454-459 (3^{rd} Edition, Pharmaceutical Press) and a decrease of pH is known to inhibit the absorption of nicotine (Fernö et al. Psychopharmacologia 1973, 31, 201-204).

Moreover, it is likely to assume that a similar absorption-enhancing effect can be seen after administration to other mucosa such as, e.g. mucosa of the nasal or gastrointestinal tract. However, in a preferred aspect of the invention, the absorption-enhancing effect is directed towards the oral mucosa.

In the present context the term “nicotine” encompasses nicotine or a nicotine derivative in any form such as, e.g., physical form like amorphous, crystalline, polymorphous etc. or chemical form like isomers and enantiomers etc as well as any salt, complex, derivative or solvate thereof. Nicotine may be selected from nicotine base nicotine hydrochloride, nicotine dihydrochloride, nicotine monotartrate, nicotine bitartrate, nicotine sulfate, nicotine zinc chloride such as nicotine zinc chloride monohydrate and nicotine salicylate, or it may be selected from nicotine resins such as nicotine polacrilex or e.g. a nicotine-cellulose or cellulose derivative adduct including MCC-nicotine (e.g. nicotine sorbed on microcrystalline cellulose as described in WO 2004/056363).

Although the present invention primarily provides compositions suitable for use for administration to the oral mucosa and moreover such compositions normally are in liquid form, it is contemplated that the findings with respect to increased absorption of nicotine from the composition and/or the route of administration between the gum and the cheek in the same manner apply to administration to other mucosa. Accordingly, whenever relevant, nicotine may be used in the form of a nicotine-carrier adduct or complex such as will be described in the following. However, a particular interesting composition of the invention is in a form that is suitable for administration to the oral mucosa and in a delivery device that is constructed to deliver the composition to an application site between the gum and the cheek or between the gum and the lips. Such compositions are normally in liquid form including solutions.

In some embodiments a composition of the invention comprises a carrier for nicotine such as mentioned above. The carrier may be a cellulose such as a microcrystalline cellulose (“mcc”). The microcrystalline cellulose may be synthetic or semi-synthetic celluloses, or it may be derived from natural celluloses. Certain specific embodiments may also utilize other forms of carriers, in addition to or including mcc, such as but not limited to fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. or mixtures thereof. While not intended to be bound by theory, it is believed as of the time of this patent application that nicotine may interact the carrier (for example, mcc or other suitable carrier including other cellulose carriers) by absorbing into and/or adsorbing onto the carrier. Such interaction is completely or nearly completely reversible.

The microcrystalline cellulose may be selected from the group consisting of AVICEL® grades PH-100, PH-102, PH-103, PH-105, PH-112, PH-113, PH-200, PH-300, PH-302, VIVACEL®) grades 101, 102, 12, 20 and EMOCEL® grades 50M and 90M, and the like, and mixtures thereof.

Suitable carriers may also be those disclosed in WO 2004/064811, which is hereby included by reference.

More specifically, it is contemplated that a relatively high surface area may be of importance for a carrier that is suitable for use. Accordingly, the specific surface area of suitable carriers is normally at least 0.7 m²/g such as, e.g., 1 m²/g. In certain uses the specific surface area may range between about 0.7 m²/g and at least about 100 m²/g and/or may be anything within this range and/or may be any mixture of sizes within this range. For example, in certain embodiments, the surface area may be about 0.7 m²/g, about 1 m²/g, about 1.5 m²/g, about 2.0 m²/g, about 3.0 m²/g, about 5 m²/g, about 7 m²/g, about 10 m²/g, about 15 m²/g, about 20 m²/g, about 25 m²/g, about 35 m²/g, about 45 m²/g, about 50 m²/g, about 75 m²/g, about 100 m²/g and above about 100 m²/g, or combinations thereof. Such carriers having such suitable surface areas may include, but are not limited to, mcc, fibrous material or carbohydrates including cellulose (including hemicellulose, celluloses with different crystallinities and structures (e.g., varying structures including solid fibers, and addition or including fibers or the like in various structures such as web-like structures and/or other structures), including naturally occurring celluloses including Cladophora sp. Algae cellulose or the like), dextran, agarose, agar, pectin, alginate, xanthan, chitosan, starch (including potato starch, shoti starch) etc. and/or mixtures thereof.

In a specific embodiment, nicotine is sorbed on microcrystalline cellulose. In general, the mean particle size of the carrier such as microcrystalline cellulose is one that is not too low and neither too high such as, e.g., at the most about 500 μm, at the most about 450 μm, at the most about 300 μm, or at the most about 200 μm, or from about 5 to about 500 μm, from 10 to about 500 μm, from 15 to about 500 μm, from about 20 to about 500μm, from about 30 to about 500 μm, from about 40 to about 500 μm, from about 10 to about 400 pm, from about 20 to about 400 μm, from about 30 to about 400 μm, from about 40 to about 400 μm, from about 30 to about 300 μm, from about 40 to about 300 μm, from about 50 to about 250 μm, from about 50 to about 200 μm or from about 75 to about 200 μm. In specific embodiments the particle size used were about 100 μm. In a preferred aspect, the mean particle size is in a range of from about 15 to about 250 μm such as from about 20 to about 200 μm. In the examples herein a quality of microcrystalline cellulose having a mean particle size of 180 μm has proved to be well-suited for the present purpose.

In an embodiment a composition according to the invention contains nicotine as a nicotine-microcrystalline cellulose carrier complex in which said nicotine is at least partly sorbed on microcrystalline cellulose and/or is at least partially absorbed into the carrier and/or is at least partially adsorbed onto the carrier (e.g., mcc), or mixtures thereof. Such interaction is completely or nearly completely reversible.

Hence, in certain specific embodiments nicotine is sorbed on microcrystalline cellulose, absorbed into the mcc and/or adsorbed onto the mcc, and/or combinations thereof.

In embodiments of the present invention, the carrier (e.g., but not limited to mcc and/or other naturally-occurring cellulose) is at least partially porous. This porosity may be due, for example but not limited to, the structure of the carrier, for example, branched, fibrous, or weblike structures may have pores. Ranges of pore sizes include but are not limited to pore volumes of about 0.01 cm³/g and include, but are not necessarily limited to pore volume ranges of from about 0.003 cm³/g or less to about 0.025 cm³/g, to about or greater than 0.60 cm³/g.

In general, the nicotine carrier complex or nicotine carrier adduct is present in a composition of the invention in a concentration of at least about 2% w/w such as in a range from about 2% w/w to about 98% w/w, from about 2% to about 96% w/w, from about 2% w/w to about 95% w/w, from about 3% w/w to about 90% w/w, from about 4% w/w to about 85% w/w, from about 5% w/w to about 80% w/w, from about 5% w/w to about 75% w/w, from about 5% w/w to about 70% w/w, or from about 7.5% w/w to about 65% w/w.

In certain embodiments, the amount of nicotine sorbed, for example absorbed into and/or adsorbed onto to carrier can be up to 50% or more of the total weight of the composition. Ranges of the amount of nicotine sorbed onto the carrier in the present invention range for less than about 1% of the total weight of the composition to more than about 50% of the composition, including all amounts within this range. While applicants do not intend the invention to be bound by theory, it is believed at the time of preparing this application that the maximum amount of nicotine that can be sorbed onto and/or into the carrier, thereby affecting the amount, for example the percent nicotine by weight of the total composition (e.g., the maximum percentage) is affected by properties of the carrier, including but not limited to the structure of the carrier, the porosity of the carrier, and the surface area of the carrier.

In certain embodiments, the concentration of the nicotine carrier complex or nicotine carrier adduct in a composition of the invention is present in a concentration such as, e.g., from about 80% w/w to about 98% w/w, such as, e.g., from about 85% w/w to about 98% w/w, from about 90% w/w to about 98% w/w, from about 92% w/w to about 98% w/w, from about 93% w/w to about 97% w/w or from about 94% w/w to about 96% w/w.

The data presented herein, indicate the relative concentration of the artificial sweetener and nicotine, respectively, to be important for the rate of nicotine-absorption in the bloodstream as exemplified in examples 5 and 6, wherein saccharin sodium is used as artificial sweetener (FIG. 3 and FIG. 4). Accordingly, the present invention relates to using an artificial sweetener, or a salt, complex, derivative or solvate thereof, in a weight ratio of from at least about 0.2 such as, e.g., at least about 0.3, at least about 0.4 or at least about 0.5, wherein the weight ratio is calculated as the ratio between the artificial sweetener and nicotine in the form of the free base nicotine. Moreover, or alternatively, the artificial sweetener is used in a weight ratio of at least about 0.2 and at the most about 5, wherein the weight ratio is calculated as stated before. In a specific embodiment, the artificial sweetener is saccharin, or a salt, complex, derivative or solvate thereof, and it is used in a weight ratio of from about 0.2 to about 2, such as, from about 0.3 to about 1.8, from about 0.4 to about 1.6, from about 0.5 to about 1.5, from about 0.6 to about 1.4, from about 0.7 to about 1.3, from about 0.8 to about 1.2, from about 0.9 to about 1.1, wherein the weight ratio is calculated as the ratio between saccharin in the form of its sodium salt and nicotine in the form of the free base nicotine. The weight ratios mentioned above relates to situations where the sweetener is in the form of saccharin sodium. Based on the molecular weight of saccharin sodium and that of another relevant artificial sweetener, a person skilled in the art can easily recalculate the above-mentioned ratios to other relevant artificial sweeteners or to other forms of saccharin (i.e. in the form of the free acid or in other salt forms). Accordingly, an artificial sweetener, or a salt, complex, derivative or solvate thereof, is used according to the invention in a molar ratio relative to nicotine of from at least about 0.1 such as, e.g., at least about 0.2, at least about 0.25 or at least about 0.3. Alternatively, the artificial sweetener is used in a molar ratio relative to nicotine of at least about 0.1 and at the most about 3.5, such as, e.g., from about 0.1 to about 1.4, such as, from about 0.2 to about 1.2, from about 0.3 to about 1.1, from about 0.35 to about 1, from about 0.4 to about 0.9, from about 0.5 to about 0.8, from about 0.5 to about 0.75, from about 0.6 to about 0.7.

The absorption enhancing effect of the artificial sweetener used according to the invention is obtained by administration of an artificial sweetener agent and nicotine via mucosa such as, e.g., oral, nasal, gastrointestinal mucosa or via the skin. In a preferred aspect, the administration is to the oral mucosa.

As demonstrated in the Examples herein a suitable absorption enhancing effect is obtained by use of the artificial sweetener together with nicotine in the same composition. However, the absorption enhancing effect may also be effective in cases where the artificial sweetener is administered before or after administration of nicotine. However, it is believed that it is of importance that the same administration route is employed in case of sequential administration.

Furthermore, in order to ensure the best conditions for a fast on-set of action of nicotine it is preferred that the artificial sweetener and nicotine is administered in liquid form i.e. either in the form of a solution, dispersion, emulsion or suspension or the like. In a preferred aspect, the administration is in the form of an aqueous solution or a water-soluble organic solvent based solution.

In those cases where a liquid is administered, the concentration at which the artificial sweetener, or a salt, complex, derivative or solvate thereof, is used as an absorption enhancing agent for a nicotine is preferably from about 20 mM to about 200 mM, such as, e.g., from about 20 mM to about 160 mM, from about 30 mM to about 120 mM, from about 40 mM to about 80 mM, from about 50 mM to about 70 mM, or more preferably from about 56 mM to about 60 mM. In a preferred embodiment the artificial sweetener is saccharin sodium, which is preferably used in a concentration from about 0.5% (w/v) to about 5% (w/v), such as, e.g., from about 0.7% (w/v) to about 4% (w/v), from about 0.9% (w/v) to about 3% (w/v), from about 1.1% (w/v) to about 2% (w/v), from about 1.3% (w/v) to about 1.7% (w/v), or more preferably from about 1.4% (w/v) to about 1.5% (w/v).

In those cases where the artificial sweetener and/or the nicotine is administered in solid or semi-solid form (e.g. in the form of a powder spray, a bioadhesive patch or the like), the weight-weight percentage concentration of the artificial sweetener in the composition may correspond to the above values given in weight-volume percentage, but the concentration may also be much higher (such as, e.g., up to about 50% w/w)

According to the present invention the above concentrations of artificial sweetener, or a salt, complex, derivative or solvate thereof, are used in combination with a concentration of nicotine, or a salt, complex, derivative or solvate thereof, which is preferably from about 30 mM to about 300 mM, such as, e.g., from about 50 mM to about 250 mM, from about 60 mM to about 200 mM, from about 70 mM to about 150 mM, from about 80 mM to about 90 mM, or more preferably from about 84 mM to about 88 mM. In a preferred embodiment of the present invention, the concentration of nicotine, or a salt, complex, derivative or solvate thereof, is preferably from about 0.5% (w/v) to about 5% (w/v), such as, e.g., from about 0.7% (w/v) to about 4% (w/v), from about 0.9% (w/v) to about 3% (w/v), from about 1.1% (w/v) to about 2% (w/v), from about 1.3% (w/v) to about 1.7% (w/v), or more preferably from about 1.4% (w/v) to about 1.5% (w/v).

In those cases where the artificial sweetener and/or the nicotine is administered in solid or semi-solid form (e.g. in the form of a powder spray, a bioadhesive patch or the like), the weight-weight percentage concentration of nicotine in the composition may correspond to the above values given in weight-volume percentage, but the concentration may also be much higher (such as, e.g., up to about 99.5% w/w)

The artificial sweetener and the nicotine are administered to a mammal, preferably to a human, in the form of a suitable composition such as e.g. a pharmaceutical composition. The details and particulars described above relating to the use aspect apply mutatis mutandis to the composition aspect.

As mentioned above, a composition according the present invention can be provided in the form of e.g. tablets, chewing gums, lozengers, patches, inhalators, patches or oral or nasal sprays.

A person skilled in pharmaceutical formulation will know how to prepare suitable compositions using one or more pharmaceutically acceptable excipients. The nature and amount of excipients employed depend on the particular composition and its administration route. In general The pharmaceutically acceptable excipients may be e.g. fillers, binders, disintegrants, diluents, glidants, solvents, emulsifying agents, suspending agents, stabilizers, enhancers, flavours, colors, pH adjusting agents, retarding agents, wetting agents, surface active agents, preservatives etc. Details can be found in pharmaceutical handbooks such as, e.g., Remington's Pharmaceutical Science or Handbook of Pharmaceutical Excipients.

To be more specific, a composition according to the invention may comprise an additive selected from the group consisting of pH-adjusting agents, stabilizing agents, preservatives, aroma-adjusting agents, flavor-adjusting agents, coloring agents, release-adjusting agents, complexing agents including EDTA. Furthermore, it may contain a buffer substance. Suitable buffer substances for use in the present context are e.g. alkaline or alkaline earth metal salts such as, e.g., sodium hydroxide, potassium hydroxide, magnesium hydroxyde, carbonates including monocarbonate, bicarbonate and sesquicarbonate such as, e.g, sodium carbonate, sodium hydrogen carbonate, potassium carbonate, potassium hydrogen carbonate, magnesium carbonate, calcium carbonate etc., glycinate, phosphate including monohydrogenphosphate, dihydrogenphosphate and trihydrogenphosphate, glycerophosphate, gluconate, borate, ammonium, and mixtures thereof salts of organic or inorganic acids such as, e.g., acetates, citrates, tartrates etc.

In those cases where the composition is designed for oral administration including administration to the mouth cavity (e.g. in the form of liquid, solid or semi-solid composition such as, e.g., in the form of a chewing gum or lozenge including pastils, toffees, hard boilies and other candy-like compositions) one or more sweeteners may be incorporated in the compositon. Suitable sweeteners are selected from the group consisting of mono-, di-, tri- and polysaccharides, artificial sweeteners such as those having a sulfonamide group, such as saccharine or Ace-K, and natural and synthetic non-saccharide-based sweeteners. In specific embodiments, the sweetener is isomalt, xylitol or sorbitol, or combinations thereof.

In other embodiments, a composition according to the invention may comprise a further therapeutically and/or prophylactically active substance.

In a preferred embodiment of the present invention, the composition is in liquid form. Such composition can be used for administration via mucosa, such as, e.g., via the oral mucosa. In a specific embodiment of the present invention, the composition is provided in the form of an oral spray.

In one embodiment the composition of the present invention comprises
i) saccharin or a salt, complex, derivative or solvate thereof,
ii) nicotine or a salt, complex, derivative or solvate thereof,
iii) a solvent
iv) a viscosity-increasing agent
v) optionally a taste-masking agent.

In certain embodiments, the solvent mentioned in item iii) in the above can be, for example, any alcohol selected from the group consisting of ethanol, propanol, isopropanol, preferably ethanol. The concentration of the solvent, for example alcohol, can be from about 60% (v/v) to about 95% (v/v), such as, e.g., from about 70% (v/v) to about 90% (v/v), from about 75% (v/v) to about 85% (v/v). Preferably the concentration of alcohol in the composition of the present invention is about 80% (v/v).

The viscosity increasing agent mentioned in item iv) in the above can be any viscosity increasing agent suitable for use in fluid compositions. In a preferred embodiment, the viscosity increasing agent is glycerin. The concentration of the viscosity increasing agent, for example glycerin, can be from about 10% (w/v) to about 20% (w/v), such as, from about 12% (w/v) to about 18% (w/v), from about 14% (w/v) to about 16% (w/v).

In order to alleviate any possible unpleasant taste of the composition according to the present invention, a taste-masking agent can optionally be included. Such taste-masking agents can be selected from the group consisting of but not limited to peppermint oil, cinnamom, liquorice, citrus and spearmint, preferably peppermint oil. The concentration of the taste-masking agent, for example, peppermint oil can be from about 1% (w/v) to about 5% (w/v), such as, e.g., from about 2% (w/v) to about 5% (w/v), from about 3% (w/v) to about 4% (w/v), from about 3.5% (w/v) to about 3.6% (w/v).

In a specific embodiment the present invention provides a composition comprising
i) saccharin sodium in a concentration of from about 1% (w/v) to about 2% (w/v),
ii) nicotine in a concentration of from about 1% (w/v) to about 2% (w/v),
iii) ethanol in a concentration of from about 75% (v/v) to about 90% (v/v),
iv) glycerine in a concentration of from about 12% (w/v) to about 18% (w/v)
v) optionally peppermint oil in a concentration of from about 2% (w/v) to about 5% (w/v), with the proviso that the total concentration is 100% w/v.

In another specific embodiment the present invention provides a composition comprising
i) saccharin sodium in a concentration of from about 1.3% (w/v) to about 1.7% (w/v),
ii) nicotine in a concentration of from about 1.3% (w/v) to about 1.7% (w/v),
iii) ethanol in a concentration of from about 80% (v/v) to about 85% (v/v),
iv) glycerine in a concentration of from about 14% (w/v) to about 16% (w/v)
v) optionally peppermint oil in a concentration of from about 3% (w/v) to about 4% (w/v), with the proviso that the total concentration is 100% w/v.

The above-mentioned compositions are preferably in the form of an oral or mouth spray intended to be applied to the oral mucosa. Normally, a suitable volume to be applied is in a range of from about 50 to about 150 μl such as, e.g., from about 50 to about 100 μl. In the examples herein the volume applied is 70 μl.

The normal dose of nicotine administered is in a range of from about 1 to about 2.5 mg, normally 2 mg provided by 2×70 μl.

Recommended daily dosage is at the most about 30 doses of 2×70 μl.

As mentioned in the above, the present invention provides a composition, the administration of which improves the rate of nicotine-absorption in the bloodstream. The provided improvement relates both to the rate of nicotine-absorption in the bloodstream and to the attained maximum plasma-concentrations of nicotine in the bloodstream (see, for example, Example 7).

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Composition A

Composition A was prepared so that 70 μl of the composition contains:

| | | |
|---|---|---|
| Nicotine | 1.00 | mg |
| Glycerine | 10.588 | mg |
| Saccharin Sodium | 1.00 | mg |
| Peppermint oil | 2.50 | mg |
| Ethanol 99.5% | 55.931 | μl |
| (undenatured ethanol) | (44.30 | μg) |
| Water | 1.739 | μl |
| (purified water) | (1.74 | μg) |

### Example 2

### Process for Preparation of Composition A

For preparation of 16 liters of composition A, 10190 g Ethanol 99.5%, 400 g water, 2440 g glycerine, 230 g saccharin sodium and 570 g peppermint oil were mixed in a stainless steel container and stirred until complete dissolution.

Then 230 g nicotine was added and the resultant solution was stirred for additional 15 minutes.

### Example 3

### Composition B

Composition B was prepared so that 70 μl of the composition contains:

| | | |
|---|---|---|
| Nicotine | 1.00 | mg |
| Glycerine | 11.088 | mg |
| Saccharin Sodium | 0.5 | mg |
| Peppermint oil | 2.50 | mg |
| Ethanol 99.5% | 55.931 | μl |
| (undenatured ethanol) | (44.30 | μg) |
| Water | 1.739 | μl |
| (purified water) | (1.74 | μg) |

### Example 4

### Process for Preparation of Composition B

For preparation of 16 liters of composition B, 10190 g Ethanol 99.5%, 400 g water, 2440 g glycerine, 115 g saccharin sodium and 570 g peppermint oil were mixed in a stainless steel container and stirred until complete dissolution.

Then 230 g nicotine was added and the resultant solution was stirred for additional 15 minutes.

### Example 5

### Blood-stream Nicotine Concentrations After Administration of Composition A

70 μl of composition A was sprayed on the mucosal side of each cheek (i.e. a total dose of 2×70 μl). A suitable time intervals a blood sample was drawn and the plasma concentration of nicotine was determined by a capillary column gas chromatography method specific for nicotine (i.e. nicotine is separated from its major metabolite).

### Example 6

### Blood-stream Nicotine Concentrations After Administration of Composition B

The procedure was as described in Example 5.

### Example 7

### Comparison of Blood-stream Nicotine Concentrations After Administration of Quit and Composition A, Respectively

The procedure was as described in Example 5. As comparison was used the Quit formulation in a dose of 2 mg of nicotine.

### LEGENDS

FIG. 1: Plasma levels of nicotine (ng/ml) at different times after administration of commercially available products as described in WO 2004/056363.

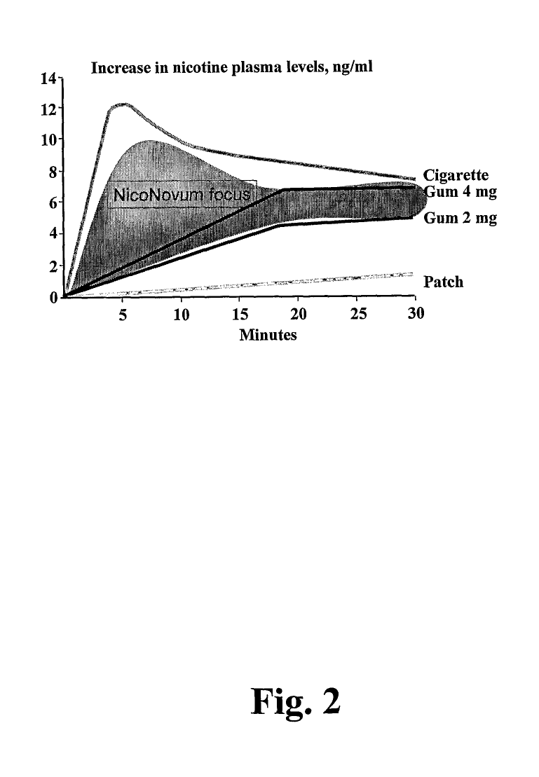

FIG. 2: Illustration of the focus area of NicoNovum with respect to the rate of nicotine-absorption in the bloodstream and to the attained maximum plasma-concentrations of nicotine in the bloodstream.

FIG. 3: Plasma levels of nicotine (ng/ml) at different times after administration of a composition according to the present invention compared to plasma levels of nicotine (ng/ml) at different times after administration of Quit. Two sprayings of a composition according to the present invention, each spraying containing 1 mg of nicotine and I mg of saccharin was administered to two different human subjects. Plasma levels of nicotine reach a maximum of 8.3-11.1 ng nicotine per ml plasma after 5-10 minutes upon administration of the composition according to the present invention. After 10 min the plasma levels of nicotine are approximately twice as high when a composition according to the present invention was administered as when Quit was administered.

FIG. 4: Plasma levels of nicotine (ng/ml) at different times after administration of two sprayings each containing 1mg of nicotine and 0.5 mg of saccharin to two different human subjects. Plasma levels of nicotine reach a maximum of 6.3-6.5 ng nicotine per ml plasma after 5-10 minutes.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

1. A method of enhancing an absorption of a nicotine or a salt, complex, derivative or solvate thereof, by a subject comprising the step of administering a sufficient amount of an artificial sweetener comprising a sulfonamide group, or a salt, complex, derivative or solvate thereof, to enhance the absorption of the nicotine, or a salt, complex, derivative or solvate thereof.

2. The method according to claim 1, wherein the artificial sweetener is saccharin or acesulfame or a salt, complex, derivative or solvate thereof.

3. The method according to claim 2, wherein the artificial sweetener is saccharin or a salt, complex, derivative or solvate thereof.

4. The method according to claim 3, wherein the artificial sweetener is saccharin or an alkaline salt thereof.

5. The method according claim 3, wherein the artificial sweetener is saccharin, or a salt, complex, derivative or solvate thereof, and is used in a weight ratio of from at least about 0.2, and wherein the weight ratio is calculated as the ratio between saccharin in the form of its sodium salt and nicotine in the form of the free base nicotine.

6. The method according to claim 5, wherein the saccharin, or a salt, complex, derivative or solvate thereof, is used in a weight ratio of at least about 0.2 and at the most about 5.

7. The method according to claim 5, wherein the saccharin, or a salt, complex, derivative or solvate thereof is used in a weight ratio of from about 0.2 to about 2.

8. The method according to claim 4, wherein the artificial sweetener is saccharin sodium.

9. The method according to claim 1, wherein the absorption enhancing effect is obtained by administration of an artificial sweetener agent and nicotine via mucosa.

10. The method according to claim 9, wherein the mucosa is the oral mucosa.

11. The method according to claim 10, wherein the administration of the artificial sweetener and the nicotine is substantially simultaneous or sequentially.

12. The method according to claim 11, wherein the administration is substantially simultaneous.

13. The method according claim 9, wherein an aqueous solution comprising the artificial sweetener and nicotine is administered.

14. A composition comprising an artificial sweetener comprising a sulfonamide group, or a salt, complex, derivative or solvate thereof, and nicotine or a salt, complex, derivative or solvate thereof, wherein the artificial sweetener and the nicotine is present in a weight ratio of from at least about 0.2, and the weight ratio is calculated as the ratio between the artificial sweetener in the form of its sodium salt and nicotine in the form of the free base nicotine.

15. A composition according to claim 14, wherein the weight ratio is at the most about 5.

16. A composition according to claim 15, wherein the weight ratio is from about 0.2 to about 2.

17. A composition according to any of claim 14, wherein the concentration of artificial sweetener, or a salt, complex, derivative or solvate thereof, is from about 0.5% (w/v) to about 5% (w/v).

18. A composition according to claim 17, wherein the concentration of artificial sweetener, or a salt, complex, derivative or solvate thereof, is from about 1.4% (w/v) to about 1.5% (w/v).

19. A composition according to claim 14, wherein the concentration of nicotine, or a salt, complex, derivative or solvate thereof, is from about 0.5% (w/v) to about 5% (w/v).

20. A composition according to claim 19, wherein the concentration of nicotine, or a salt, complex, derivative or solvate thereof, is from about 1.4% (w/v) to about 1.5% (w/v).

21. A composition according to claim 14, wherein the artificial sweetener is saccharin or a salt, complex, derivative or solvate thereof.

22. A composition according to claim 14, wherein the artificial sweetener is saccharin sodium.

23. A composition according to claim 14 in liquid form.

24. A composition according to claim 14 for administration to the oral mucosa.

25. A composition according claim 14 comprising i) saccharin or a salt, complex, derivative or solvate thereof, ii) nicotine or a salt, complex, derivative or solvate thereof, iii) a solvent.

26. A composition according to claim 25, wherein the solvent is an alcohol selected from the group consisting of ethanol, propanol, isopropanol and combinations thereof.

27. A composition according to claim 26, wherein the concentration of alcohol is from about 60% (v/v) to about 95% (v/v).

28. A composition according to claim 27, wherein the concentration of alcohol is about 80% (v/v).

29. A composition according to claim 25 containing a viscosity increasing agent such as glycerin.

30. A composition according to claim 29, wherein the concentration of glycerin is from about 10% (w/v) to about 20% (w/v).

31. A composition according to claim 25, wherein a taste-masking agent is present.

32. A composition according to claim 31, wherein the taste-masking agent is selected from the group consisting of peppermint, cinnamom, liquorice, citrus, spearmint and combinations thereof.

33. A composition according to claim 32, wherein the taste-masking agent is peppermint.

34. A composition according to claim 33, wherein the concentration of peppermint is from about 1% (w/v) to about 5% (w/v).

35. A composition according to claim 14 containing
i) saccharin sodium in a concentration of from about 1% (w/v) to about 2% (w/v),
ii) nicotine in a concentration of from about 1% (w/v) to about 2% (w/v),
iii) ethanol in a concentration of from about 75% (v/v) to about 90% (v/v),
iv) glycerine in a concentration of from about 12% (w/v) to about 18% (w/v)
v) optionally peppermint oil in a concentration of from about 2% (w/v) to about 5% (w/v),
with the proviso that the total concentration does not exceed 100% w/v.

36. A composition according to claim 14 containing i) saccharin sodium in a concentration of from about 1.3% (w/v) to about 1.7% (w/v), ii) nicotine in a concentration of from about 1.3% (w/v) to about 1.7% (w/v), iii) ethanol in a concentration of from about 80% (v/v) to about 85% (v/v), iv) glycerin in a concentration of from about 14% (w/v) to about 16% (w/v) v) optionally peppermint oil in a concentration of from about 3% (w/v) to about 4% (w/v), with the proviso that the total concentration does not exceed 100% w/v.

37. A composition according to claim 14 in the form of an oral spray.
